(19) United States
(12) Patent Application Publication
(10) Pub. No.: US 2025/0134728 A1
(43) Pub. Date: May 1, 2025

(54) FLUID COLLECTION ASSEMBLIES INCLUDING AT LEAST ONE INFLATION DEVICE

(71) Applicant: PUREWICK CORPORATION, Covington, GA (US)

(72) Inventors: Jason Jishen Cheng, Covington, GA (US); Eric Rehm, Greensboro, GA (US); Nicholas Austerman, Atlanta, GA (US)

(21) Appl. No.: 18/925,921

(22) Filed: Oct. 24, 2024

Related U.S. Application Data
(63) Continuation of application No. 17/447,123, filed on Sep. 8, 2021, now Pat. No. 12,156,792.
(60) Provisional application No. 63/076,474, filed on Sep. 10, 2020.

(51) Int. Cl. A61F 13/474; A61F 13/84

(52) U.S. Cl. CPC A61F 13/474; A61F 13/84; A61F 2013/8494

(57) ABSTRACT

Fluid collection assemblies, systems including the same, and methods of using the same are disclosed herein. An example fluid collection assembly includes a fluid impermeable barrier having a proximal surface and a distal surface opposite the proximal surface. The proximal surface defines an opening. The fluid collection assembly also includes at least one inflation device. The inflation device includes a bladder. The bladder may be adjacent to the distal surface of the fluid impermeable barrier or between a conduit disposed in the chamber and the distal surface. The bladder is configured to switch between a first state and at least a second state. A volume of the bladder is greater when the bladder is the second state than when the bladder is in the first state. Switching the bladder between the first and second states changes a curvature of the at least a portion of the fluid collection assembly.

### CROSS-REFERENCE TO RELATED APPLICATIONS

This application is a continuation of U.S. application Ser. No. 17/447,123 filed on Sep. 8, 2021, which claims priority to U.S. Provisional Patent Application No. 63/076,474 filed on Sep. 10, 2020, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection devices continue to seek new and improved devices, systems, and methods to collect urine.

### SUMMARY

Embodiments disclosed herein are directed to fluid collection assemblies, systems including the same, and methods of using the same are disclosed herein. In an embodiment, a fluid collection assembly is disclosed. The fluid collection assembly includes a fluid impermeable barrier defining a chamber, at least one opening, and at least one fluid outlet. The fluid impermeable barrier includes at least one proximal surface defining the at least one opening and at least one distal surface opposite the proximal surface. The fluid collection assembly also includes at least one porous material disposed in the chamber. The fluid collection assembly includes at least one inflation device that includes a bladder. The bladder is operably coupled to the fluid impermeable barrier. The bladder includes one or more walls defining at least one interior region, The bladder is configured to switch between a first state and at least a second state. A volume of the at least one interior region is greater when the bladder is in the second state than when the bladder is in the first state. Switching the bladder between the first state and the second state changes a curvature of at least a portion of the fluid impermeable barrier.

In an embodiment, a system is disclosed. The system includes a fluid collection assembly, a fluid storage container, and a vacuum source. the chamber of the fluid collection assembly, the fluid storage container, and the vacuum source are in fluid communication with each other via one or more conduits.

In an embodiment, a method of using a fluid collection assembly is disclosed. The method includes positioning at least one opening of the fluid collection assembly adjacent to a female urethral opening. The fluid collection assembly includes a fluid impermeable barrier defining a chamber, at least one opening, and at least one fluid outlet. The fluid impermeable barrier includes at least one proximal surface defining the at least one opening and at least one distal surface opposite the proximal surface. The fluid collection assembly also includes at least one porous material disposed in the chamber. The fluid collection assembly further includes at least one inflation device including a bladder and at least one valve. The bladder includes one or more walls defining at least one interior region. The method further includes flowing at least one inflation fluid through the at least one valve and into the at least one interior region of the at least one inflation device to change a curvature of the fluid impermeably barrier.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIGS. 1A and 1B are isometric views of a fluid collection assembly when a bladder of the fluid collection assembly is in a first state and a second state, respectively, according to an embodiment.

FIGS. 1C and 1D are cross-sectional schematics of the fluid collection assembly taken along planes C-C and D-D shown in FIGS. 1A and 1B, respectively, according to an embodiment.

FIG. 1E is a cross-sectional schematic of the fluid collection assembly taken along plane E-E shown in FIG. 1A, according to an embodiment.

FIGS. 2-4 are cross-sectional schematics and FIGS. 5 and 6 are side views of different fluid collection assemblies illustrating different techniques for controlling the expansion rate of the first and second regions of the bladders, according to different embodiments.

FIG. 7 is side view of a fluid collection assembly that includes a “crinkle” shaped bladder, according to an embodiment.

FIG. 8 is a side view of a fluid collection assembly that includes a plurality of inflation devices, according to an embodiment.

FIG. 9 is a schematic side view of a fluid collection system that is configured to control the shape of a fluid collection assembly, according to an embodiment.

FIGS. 10-12 are cross-sectional schematics of fluid collection assemblies having at least one inflation device positioned at locations other than an outer surface of the fluid impermeable barrier, according to different embodiments.

FIG. 13A is a cross-sectional view of a fluid collection assembly, according to an embodiment.

FIG. 13B is a cross-sectional view of the fluid collection assembly taken along plane B-B of FIG. 13A, according to an embodiment.

FIG. 14 is a block diagram of a system for fluid collection, according to an embodiment.

FIG. 15 is a flow diagram of a method to collect fluid, according to an embodiment.

### DETAILED DESCRIPTION

Embodiments are directed to fluid collection assemblies, systems including the same, and methods of using the same are disclosed herein. An example fluid collection assembly includes a fluid impermeable barrier having at least one proximal surface configured to be adjacent to an patient (i.e., an individual using the fluid collection assembly) when the fluid collection assembly is in use and at least one distal surface opposite the proximal surface. The fluid impermeable barrier at least defines a chamber, at least one opening defined by the proximal surface, and at least one fluid outlet. The fluid collection assembly also includes at least one porous material disposed in the chamber and at least one inflation device. The inflation device includes a bladder. The bladder may be adjacent to the distal surface of the fluid impermeable barrier or between a conduit disposed in the chamber and the distal surface. The bladder is configured to switch between a first state and at least a second state. A volume of the bladder is greater when the bladder is the second state than when the bladder is in the first state. Switching the bladder between the first state and the second state changes a curvature of at least a portion of the fluid impermeable barrier.

During use, the fluid collection assembly is positioned adjacent to a urethral opening (e.g., vagina) of the patient. One or more bodily fluids (e.g., urine, blood, etc.) that are discharged from the urethral opening of the patient flow through the opening and into the chamber. The bodily fluids that enter the chamber are received into the porous material and are directed towards the fluid outlet. The bodily fluids may be removed from the chamber through the fluid outlet, for example, when a suction force from a vacuum source is applied to the fluid outlet.

Generally, the fluid collection assembly conforms to the shape of the region about the urethral opening to prevent gaps between the fluid collection assembly and the region about the urethral opening. For example, bodily fluids may leak (e.g., do not enter the chamber and/or do not remain in the chamber) through gaps between the fluid collection assembly and the region about the patient. Such leaks may be embarrassing to the patient using the fluid collection assembly, create unsanitary situations, and cause the skin of the patient to remain moist which may cause skin degradation (e.g., rash) and general discomfort.

The region of the patient about the urethral opening may vary in size and shape depending on the patient. Some conventional fluid collection assemblies are manually bent to conform to the shape of the region about the urethral opening to prevent gaps between the conventional fluid collection assemblies and the region about the patient. Contact between the thighs of the patient and the conventional fluid collection assemblies maintains the bent shape of the conventional fluid collection assemblies. However, skinny patients may have too small of thighs to maintain contact with the conventional fluid collection assemblies while forgetful patients (e.g., confused patients, young children, patients with dementia) may move their legs such that the thighs no longer contact the conventional fluid collection assemblies, either of which may cause the fluid collection assembly to loss the bent shape thereof.

Embodiments of the fluid collection assemblies disclosed herein are an improvement to such fluid collection assemblies. As previously discussed, the fluid collection assemblies disclosed herein include at least one inflation device. The inflation device is configured to control the curvature of at least a portion of the fluid impermeable barrier such that the fluid impermeable barrier generally conforms to the shape (e.g., curvature) of the region about the urethral opening. In other words, the inflation device allows the fluid collection assembly to maintain the shape thereof without relying on contact between the fluid collection assembly and the thighs of the patient. As such, the inflation device may minimize gaps between the fluid impermeable barrier and the region about the urethral opening through which bodily fluids may leak. For example, the inflation device includes a bladder that is configured to switch between a first state and at least a second state. The bladder switches from the first state to the second state by increasing a volume thereof and from the second state to the first state by decreasing the volume thereof. The bladder may increase and decrease the volume thereof by adding or removing, respectively, at least one inflation fluid (e.g., air, water, saline, or other suitable fluid) from the bladder. A curvature of the inflation device may change when switching the bladder between the first and second states thereof. For example, the bladder may exhibit a first shape (e.g., first curvature) when the bladder is in the first state and a second shape (e.g., second curvature) when the bladder is in the second state, wherein the first shape and the second shape are different. The shape of at least a portion of the rest of the fluid collection assembly (e.g., fluid impermeable barrier) generally corresponds to the shape of the bladder and, thus, the inflation device may control the shape of the fluid impermeable barrier.

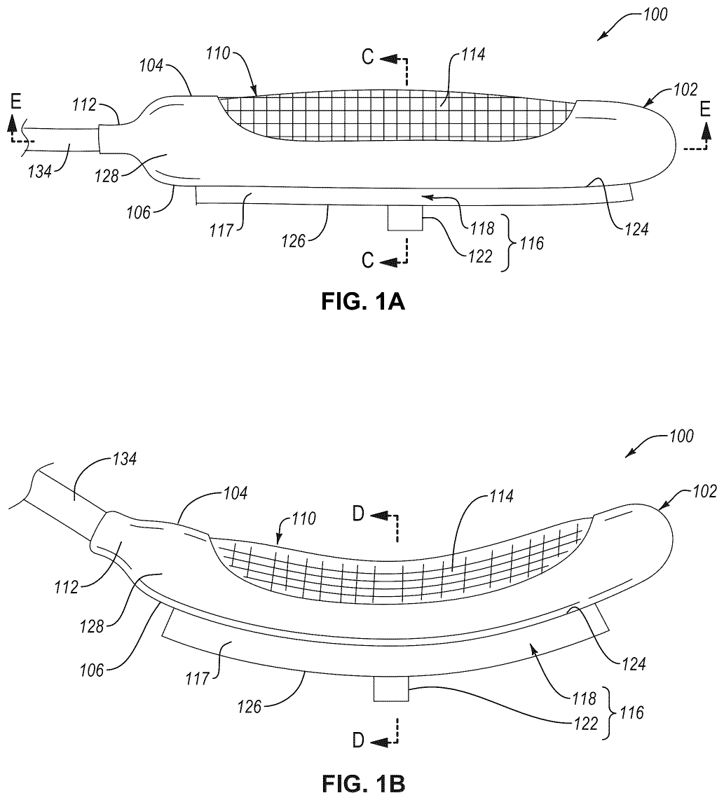

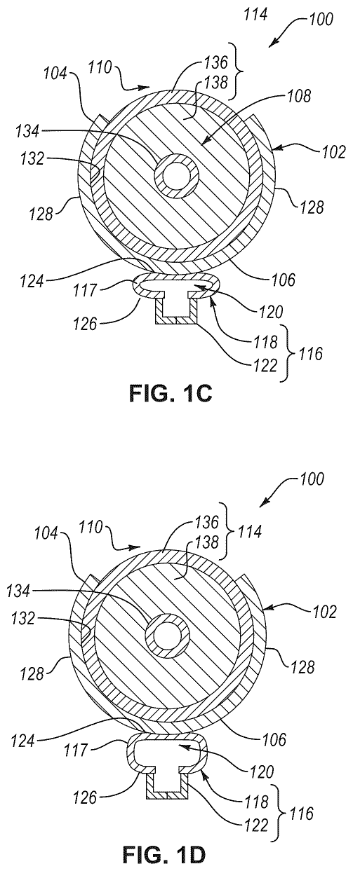

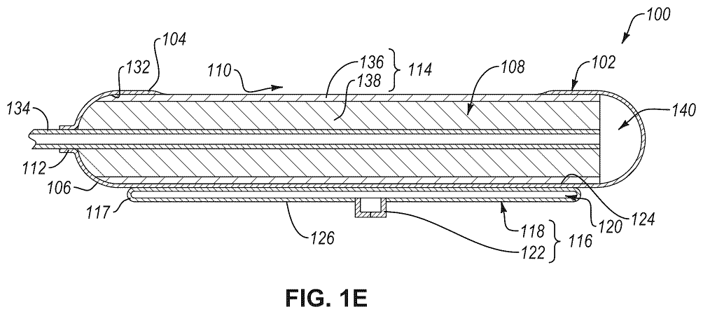

FIGS. 1A and 1B are isometric views of a fluid collection assembly 100 when a bladder 118 of the fluid collection assembly 100 is in a first state and a second state, respectively, according to an embodiment. FIGS. 1C and 1D are cross-sectional schematics of the fluid collection assembly 100 taken along planes C-C and D-D shown in FIGS. 1A and 1B, respectively, according to an embodiment. FIG. 1E is a cross-sectional schematic of the fluid collection assembly 100 taken along plane E-E shown in FIG. 1A, according to an embodiment. The fluid collection assembly 100 includes a fluid impermeable barrier 102. The fluid impermeable barrier 102 includes a proximal surface 104 that is configured to be positioned adjacent to an patient during use and a distal surface 106 opposite the proximal surface 104. The fluid impermeable barrier 102 defines at least a chamber 108, at least one opening 110 that is defined by the proximal surface 104, and at least one fluid outlet 112. The fluid collection assembly 100 also includes at least one porous material 114 disposed in the chamber 108 and at least one inflation device 116 adjacent to the distal surface 106.

The inflation device 116 includes a bladder 118. The bladder 118 includes one or more walls 117 defining an interior region 120. The inflation device 116 also includes at least one valve 122 in fluid communication with the interior region 120. The valve 122 is configured to selectively permit flow of an inflation fluid into and/or out of the interior region 120. For example, the valve 122 may allow an inflation fluid to enter the interior region 120 when it is desirable to increase the volume of the bladder 118 which, in turn, changes a shape (e.g., curvature) of the bladder 118 and at least a portion of the fluid impermeable barrier 102. The valve 122 may also enable removing the inflation fluid from the interior region 120 when it is desirable to decrease the volume of the bladder 118 which, in turn changes a shape (e.g., return to the initial shape) of the bladder 118 and at least a portion of the fluid impermeable barrier 102.

Adding or removing the inflation fluids into and from the interior region 120 changes the state of the bladder 118. The bladder 118 may exhibit at least a first state and a second state. The amount (volume or weight) of inflation fluids present in the interior region 120 is greater when the bladder 118 is in the second state than when the bladder 118 is in the first state. In an example, as shown in FIG. 1C, the bladder 118 is in the first state when the bladder 118 is in a deflated state (e.g., there are not or substantially no fluids in the interior region 120). However, it is noted that the bladder 118 may be in the first state when some inflation fluids are present in the interior region 120. Generally, the fluid collection assembly 100 is provided without any inflation fluids in the interior region 120 (e.g., the fluid collection assembly 100 is provided with the bladder 118 in the deflated state) thereby preventing the bladder 118 from leaking during shipping and handling and inhibiting contaminant growth (e.g., bacteria growth) in the interior region 120 before use. In an example, as shown in FIG. 1D, the bladder 118 is in the second state when the bladder 118 is in an at least partially inflated state. The fluid collection assembly 100 is generally not provided with the bladder 118 in the second state since the inflation fluids may leak during shipping and handling and the inflation fluids may encourage contaminant growth more than if the bladder 118 was provided in an deflated state. However, in some examples, the fluid collection assembly 100 is provided in the second state.

The bladder 118 may exhibit one or more additional states (e.g., a third state, a fourth state, and so forth) besides the first and second states discussed above. In an embodiment, the one or more additional states may include less inflation fluids in the interior region 120 (e.g., is more deflated) than the first state (e.g., the first state is a partially inflated state). In such an embodiment, the one or more additional states may include a deflated or partially deflated state and may be formed by removing inflation fluids from the interior region 120 when the bladder 118 is in the first or second state. In an embodiment, the one or more additional states may include more inflation fluids in the interior region 120 (e.g., is more inflated) than the first state (e.g., the first state is a deflated or partially inflated state) and include less inflation fluids in the interior region 120 than the second state. In such an embodiment, the one or more additional states include a partially inflated state and may be formed by adding or removing inflation fluids to the interior region 120 when the bladder 118 is in the first state or second state, respectively. In an embodiment, the one or more additional states may include more inflation fluids in the interior region 120 than the second state (e.g., the second state is a partially inflated state). In such an embodiment, the one or more additional states may be an at least partially inflated state and may be formed by adding inflation fluids to the interior region 120 when the bladder 118 is in the first state or second state. It is noted that, in some embodiments, the bladder 118 may only include the first and second states.

As shown in FIGS. 1A and 1B, switching the bladder 118 between the first state and the second state (or any of the other states thereof) changes the shape of the bladder 118. As discussed herein, changing the shape of the bladder 118 generally refers to changing at least a curvature of the bladder 118. Changing the shape of the bladder 118 causes at least a portion of the rest of the fluid collection assembly 100, such as at least a portion of the fluid impermeable barrier 102, to change a shape thereof that generally corresponds to the shape of the bladder 118. In an embodiment, as illustrated, the bladder 118 may exhibit a generally straight shape (e.g., exhibits a radius of curvature greater than about 25 cm) when the bladder 118 is in a first state (e.g., deflated state). In such an embodiment, at least a portion of the rest of the fluid collection assembly 100 may exhibit a generally straight shape when the bladder 118 is in the first state. The bladder 118 and the fluid collection assembly 100 may exhibit the generally straight shape when the bladder 118 is in the first state, for example, when the fluid collection assembly 100 would exhibit the substantially straight shape when the inflation device 116 is omitted from the fluid collection assembly 100. For instance, the bladder 118 may be flimsy or flexible and exert substantially no force on the rest of the fluid collection assembly 100 that is sufficient to significantly modify a shape of the rest of the fluid collection assembly 100 when the bladder 118 is in the first state. Switching the bladder 118 from the first state to the second state may cause the bladder 118 to exhibit a generally curved shape (e.g., exhibits an average radius of curvature less than about 40 cm, such as less than about 35 cm, less than about 30 cm, less than about 25 cm, less than about 20 cm, less than about 15 cm, or less than about 10 cm). The curved shape of the bladder 118 may be a concave curved shape relative to the opening 110. Similarly, at least a portion of the fluid collection assembly 100 may exhibit a generally curved shape when the bladder 118 is in the second state. For example, inflating the bladder 118 may increase the rigidity of the bladder 118 such that the bladder 118 exerts a force on the rest of the fluid collection assembly 100 that is sufficient to change a shape of the rest of the fluid collection assembly 100. The radius of curvature of the bladder 118 and the fluid collection assembly 100 may be controlled based on the amount of bodily fluids that are present in the interior region 120. For example, increasing the amount of inflation fluids in the interior region 120 may decrease the average radius of curvature of the bladder 118 and the fluid collection assembly 100. Similarly, decreasing the amount of inflation fluids in the interior region 120 may increase the radius of curvature of the bladder 118. Thus, the amount of inflation fluids in the interior region 120 may control the shape of the fluid collection assembly 100 and may be used to shape the fluid collection assembly 100 to exhibit a shape that corresponds to the shape of the region about the urethral opening.

In an embodiment, not shown, the bladder 118 and the fluid collection assembly 100 may exhibit a curved shape (e.g., a radius of curvature less than about 40 cm, less than about 25 cm, etc.) when the bladder 118 is in the first state. The curved shape of the bladder 118 may be a concave curve relative to the opening 110. The bladder 118 and the fluid collection assembly 100 may exhibit the curved shape when the bladder 118 is in the first state, for example, when the fluid collection assembly 100 would exhibit the curved shape when the inflation device 116 is omitted from the fluid collection assembly 100. For instance, the bladder 118 may be flimsy and exert no force on the rest of the fluid collection assembly 100 that is sufficient to significantly modify a shape of the rest of the fluid collection assembly 100 when the bladder 118 is in the first (e.g., deflated) state. Switching the bladder 118 to the second state may either increase or decrease the radius of curvature of the bladder 118 depending on the shape that the bladder assumes when inflated. Similarly, switching the bladder 118 to the second state may either increase or decrease the average radius of curvature of the rest of the fluid collection assembly 100. Thus, the amount of inflation fluids in the interior region 120 may control the shape of the fluid collection assembly 100 and may be used to shape the fluid collection assembly 100 to exhibit a shape that corresponds to the shape of the region about the urethral opening.

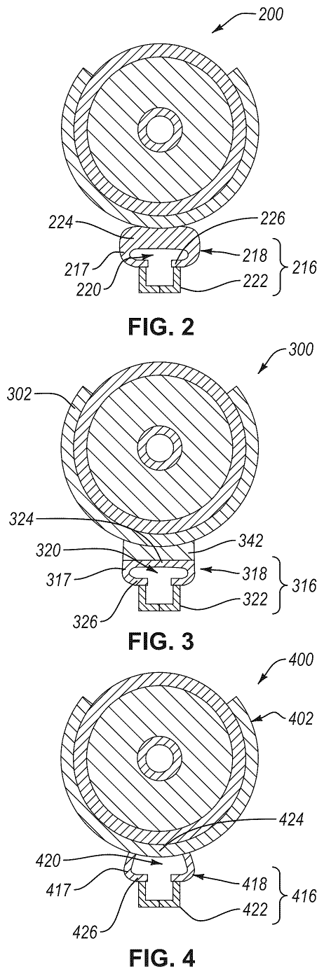

The shape that the bladder 118 assumes when switching the bladder 118 from the first state to the second state may be controlled by configuring different portions of the bladder 118 to expand at different rates (hereafter referred to as “expansion rate”). The expansion rate may be determined by taking the absolute value of the length of at least a portion (e.g., all) of bladder 118 when the bladder 118 is in the first state minus the length of the same portion of the bladder 118 when the bladder 118 is in the second state, wherein the length is measured parallel to the longitudinal axis of the fluid collection assembly 100. In an embodiment, the walls 117 of the bladder 118 includes a first region 124 and a second region 126 opposite the first region 124. The first region 124 and the second region 126 generally face the same direction as the proximal surface 104 and the distal surface 106, respectively. The first region 124 and the second region 126 may exhibit different expansion rates. In an embodiment, as illustrated, the first region 124 exhibits an expansion rate that is less than the second region 126. As such, switching the bladder 118 from the first state to the second state causes a decrease an average radius of curvature of the bladder 118 (i.e., causes the bladder 118 to bend) because a length of the second region 126 increases more than a length of the first region 124 when the bladder 118 switches from the first state to the second state. In an embodiment, the first region 124 exhibits an expansion rate that is greater than the second region 126. FIGS. 2-6 illustrate some examples of techniques that may be used to cause the first region 124 to exhibit different expansion rates than the second region 126.

The shape (e.g., curvature) that the bladder 118 assumes when in the first state, second state, and, optionally, the one or more additional states depend on a number of factors. For example, the shape that the bladder 118 assumes when the bladder 118 is in a deflated (e.g., first) state may be dictated by the shape of the rest of the fluid collection assembly 100. For instance, the bladder 118 may be formed from a relatively flexible material which allows the bladder 118 to be inflated. However, without any inflation fluids in the interior region 120, the bladder 118 merely conforms to the shape of the fluid collection assembly 100. Increasing the quantity of the inflation fluids in the interior region 120 to or near a level where the inflation fluids cause the bladder 118 to stretch may increase the rigidity of the bladder 118 to the point that the bladder 118 may exhibit a shape that is different than the fluid collection assembly 100. The shape that the bladder 118 exhibits may be, for example, an arc (e.g., a portion of a circle, oval, or other curve), one or more bends between adjacent straight or curved portions, or any other suitable shape. The different shapes that the bladder 118 may exhibit may be controlled by selecting the expansion rates of at least one of the first and second regions 124, 126, different portions of the first region 124 (e.g., one portion of the first region 124 may exhibit an expansion rate that is different than another portion of the first region 124), or different portions of the second region 126 (e.g., one portion of the second region 126 may exhibit an expansion rate that is different than another portion of the second region 126). In an example, the bladder 118 may exhibit a generally circular arc in the second state when substantially all the first region 124 exhibits the substantially same expansion rate and substantially all of the second region 126 exhibits the substantially same expansion rate, wherein the expansion rates of the first and second regions 124, 126 are different. In an example, the bladder 118 may exhibit a generally oval arc, other arc, or a bend therein when a portion of the first region 124 exhibits a different expansion rate than another portion of the first region 124 and/or when a portion of the second region 126 exhibits a different expansion rate than another portion of the second region 126. The expansion rates of the first and second regions 124, 126 may be selected based on factors discussed in more detail with regards to FIGS. 2-6.

In an embodiment, as illustrated, the inflation device 116 is positioned such that the first region 124 is adjacent to the distal surface 106 of the fluid impermeable barrier 102. Positioning the first region 124 adjacent to the distal surface 106 allows the inflation device 116 to change the shape of at least a portion of the rest of the rest of the fluid collection assembly 100. Further, a width of the bladder 118 (e.g., measured perpendicular to a length of the bladder 118 and perpendicular to thickness measured between the first and second regions 124, 126) may increase when the bladder 118 switches from the first state to the second state. However, positioning the first region 124 adjacent to the distal surface 106 may prevent the increase in the width of the bladder 118 from increasing the maximum width of the fluid collection assembly 100 when the bladder 118 switches from the first state to the second state, wherein the width of the fluid collection assembly 100 is measured perpendicular to the width of the bladder 118. The maximum width of the fluid collection assembly 100 does not change when the bladder 118 switches from the first state to the second state because the width of the bladder 118 is less than the maximum width of the fluid collection assembly 100 and/or the width of the bladder 118 does not need to significantly increase to significantly change the shape of the bladder 118. It is noted that not increasing the maximum width of the fluid collection assembly 100 when switching the bladder 118 from the first state to the second state allows the fluid collection assembly 100 to be used comfortably with patient's having average or larger than average sized thighs. For example, patients having average or larger than average thighs do not have much space between the thighs to accommodate an increased width of the fluid collection assembly 100 without uncomfortably pressing into the thighs.

The fluid impermeable barrier 102 may include at least one lateral surface 128 extending between the proximal and distal surfaces 104, 106. The lateral surface 128 may be adjacent to a thigh of an patient when the fluid collection assembly 100 is worn by the patient. In an embodiment, as discussed, above, the bladder 118 may be positioned adjacent to the distal surface 106 and not adjacent to the lateral surface 128 to prevent the bladder 118 from increasing the maximum width of the fluid collection assembly 100. In an embodiment, the bladder 118 may be positioned adjacent to the lateral surface 128 of the fluid impermeable barrier 102. In such an embodiment, switching the bladder 118 from the first state to the second state may increase the width of the fluid collection assembly 100. Increasing the width of the fluid collection assembly 100 may improve contact between smaller than average thighs and the fluid collection assembly 100. Further, when the bladder 118 is positioned adjacent to the lateral surface 128, the fluid collection assembly 100 may require two inflation devices on opposing sides of the lateral surface 128 and both inflation devices may need to receive the same amount of inflation fluids to ensure that the fluid collection assembly 100 is symmetrically shaped thereby making the manufacturing and use of the inflation device 116 more complex. It is noted that gaps may form between the fluid collection assembly 100 and the patient when the fluid collection assembly 100 is shaped non-symmetrically.

The walls 117 of the bladder 118 are formed from a material is substantially impermeable to the inflation fluid (e.g., substantially impermeable to a gas and/or a liquid) which allows the bladder 118 to retain the inflation fluids without leaks. The walls 117 may also be formed from a flexible material. The flexible material of the walls 117 allows the bladder 118 and, by extension, the fluid collection assembly 100 to change a shape thereof. For example, the flexible material of the walls 117 allow the interior region 120 to increase a volume thereof when the interior region 120 receives an inflation fluid and decrease a volume thereof when inflation fluids are removed from the interior region 120 which, in turn, changes the shape of the bladder 118. Examples of materials that may form the walls 117 of the bladder 118 include silicone, rubber, latex, polychloroprene, nylon fabric, polypropylene, polyvinyl chloride, nitrile rubber, other suitable polymers, a metal foil, a composite, or combinations thereof. It is noted that the walls 117 may contact the patient and might be formed from a biocompatible material. In an embodiment, the walls 117 are configured to stretch (e.g., elastically or plastically stretch) so the walls 117 remain taut when the bladder 118 is at least partially inflated. In an embodiment, the wall 117 forms a plurality of wrinkles when the bladder 118 is at least partially deflated and adding inflation fluid into the interior region 120 decreases the wrinkles.

The valve 122 (illustrated schematically) may include any suitable valve configured to allow for the controllable addition and removal of inflation fluids from the interior region 120. In an embodiment, the valve 122 is a luer valve and includes a male-tapper fitting or a female-taper fitting. In an embodiment, the valve 122 includes a fluid impermeable membrane with a slit or opening formed therein. The slit or opening of the fluid impermeable membrane remains substantially closed when no external load is applied thereto but opens when an external load is applied thereto (e.g., an external load caused by pressing a syringe against the fluid impermeable membrane). In an embodiment, the valve 122 may include a mechanical valve, such as a ball valve, a butterfly valve, or any other suitable mechanical valve. The mechanical valve may be manually operated or controlled using a computer. In an embodiment, the valve 122 may include a one-way valve (e.g., check valve) to limit leaks from the bladder 118 and to make the fluid collection assembly 100 easier to use. In such an embodiment, the valve 122 may only add or remove (but not both) inflation fluid from the interior region 120 and, as such, the fluid collection assembly 100 including the check valve is configured for single use. In an embodiment, the valve 122 may include a two-way valve which allows the inflation fluid to be added and removed from the interior region 120. In such an embodiment, at least a portion of the fluid collection assembly 100 (e.g., the fluid impermeable barrier 102 and the inflation device 116) may be reusable.

In an embodiment, as illustrated, the valve 122 may extend outwardly from the bladder 118. For example, the valve 122 may extend from the bladder 118 thereby allowing a user (e.g., medical practitioner or patient) of the fluid collection assembly 100 to easily access the valve 122. As shown, the valve 122 may extend a short distance only from the fluid impermeable barrier 102, such as about 2 cm or less, about 1.5 cm or less, about 1 cm or less, about 0.75 cm or less, about 0.5 cm or less, about 0.25 cm or less, or in ranges of about 0.25 cm to about 0.75 cm, about 0.5 cm to about 1 cm, about 0.75 cm to about 1.5 cm, or about 1 cm to about 2 cm. However, the valve 122 may extend a significant distance from the fluid impermeable barrier 102, such as a distance that is about 2 cm or greater, about 5 cm or greater, about 10 cm or greater, about 50 cm or greater, about 100 cm or greater, about 500 cm or greater, about 1 m or greater, about 2 m or greater, or in ranges of about 2 cm to about 10 cm, about 5 cm to about 50 cm, about 10 cm to about 100 cm, about 50 cm to about 500 cm, about 100 cm to about 1 m, or about 500 cm to about 2 m. When the valve 122 extends a significant distance from the fluid impermeable barrier 102, the valve 122 may include a flexible tube which allows a user of the fluid collection assembly 100 to easily access to the valve 122 while the fluid collection assembly 100 is positioned adjacent to the urethral opening without having the user near the urethral opening (which the patient may find uncomfortable).

In an embodiment, as illustrated, the valve 122 extends from or near a second region 126 of the bladder 118. The valve 122 at or near the second region 126 may allow a user of the fluid collection assembly 100 to access the valve 122 when the fluid collection assembly 100 is adjacent to the urethral opening since, generally, the inner thighs of the patient may contact or obstruct the proximal and lateral surfaces 104, 128 of the fluid impermeable barrier 102. Further, the valve 122 at or near the second region 126 prevents or at least inhibits the valve 122 from pressing against the inner thighs during use which may cause discomfort.

The at least one inflation fluid added or removed from the interior region 120 may include any suitable fluid, such as any suitable liquid or any suitable gas. In an embodiment, the inflation fluids are formed from a generally regarded as safe (“GRAS”) material. Forming the inflation fluids from a GRAS materials may decrease health risks caused by inadvertently exposing the patient to the inflation fluids. Examples of GRAS materials that may form the inflation fluids includes water, saline solution, alcohol solution, atmospheric air, nitrogen, any other GRAS material, or combinations thereof.

Further examples of inflation devices are disclosed in U.S. Provisional Patent Application No. 63/030,685 filed on May 27, 2020, the disclosure of which is incorporated herein, in its entirety, by this reference.

As previously discussed, the fluid collection assembly 100 includes a fluid impermeable barrier 102. In the illustrated embodiment, the fluid impermeable barrier 102 is distinct from the inflation device 116.

The fluid impermeable barrier 102 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. The fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least a surface of the fluid impermeable barrier 102 that may contact the patient may be formed from a soft and/or smooth material (e.g., silicone), thereby reducing chaffing. In an embodiment, the fluid impermeable barrier 102 may be formed from a flexible material, such as silicone, which allows the fluid impermeable barrier 102 to be bent into a shape that conforms the anatomy of the patient. Further, as shown in FIGS. 1B and 1C, forming the fluid impermeable barrier 102 from a flexible material allows the fluid impermeable barrier 102 to accommodate the shape and/or size changes by switching the fluid collection assembly 100 and the bladder 118 between states.

In some examples, the fluid impermeable barrier 102 may be tubular (ignoring the opening), such as substantially cylindrical (as shown), oblong, prismatic, or flattened tubes. During use, the outer surface (e.g., at least a portion of one or more of the proximal surface 104, the distal surface 106, or the lateral surface 128) of the fluid impermeable barrier 102 may contact the patient. The fluid impermeable barrier 102 may be sized and shaped to fit in the gluteal cleft between the legs of a female user when the bladder 118 are in at least the second state.

The opening 110 provides an ingress route for fluids to enter the chamber 108. The opening 110 may be defined by the fluid impermeable barrier 102 such as by an inner edge of the fluid impermeable barrier 102. For example, the opening 110 is formed in and extends through the fluid impermeable barrier 102, from the proximal surface 104 to an inner surface 132 of the fluid impermeable barrier 102, thereby enabling bodily fluids to enter the chamber 108 from outside of the fluid collection assembly 100. The opening 110 may be an elongated hole in the fluid impermeable barrier 102. For example, the opening 110 may be defined as a cut-out in the fluid impermeable barrier 102. The opening 110 may be located and shaped to be positioned adjacent to a female urethral opening.

The fluid collection assembly 100 may be positioned proximate to the female urethral opening and urine or other bodily fluids may enter the chamber of the fluid collection assembly 100 via the opening 110. The fluid collection assembly 100 is configured to receive the bodily fluids into the chamber 108 via the opening 110. When in use, the opening 110 may have an elongated shape that extends from a first location below the urethral opening (e.g., at or near the anus or the vaginal opening) to a second location above the urethral opening (e.g., at or near the top of the vaginal opening or the pubic hair).

The opening 110 may have an elongated shape because the space between the legs of a female is relatively small when the legs of the female are closed, thereby only permitting the flow of the bodily fluids along a path that corresponds to the elongated shape of the opening 110 (e.g., longitudinally extending opening). The opening 110 in the fluid impermeable barrier 102 may exhibit a length measured along the longitudinal axis of the fluid collection assembly 100 that may be at least about 10% of the length of the fluid collection assembly 100, such as about 25% to about 50%, about 40% to about 60%, about 50% to about 75%, about 65% to about 85%, or about 75% to about 95% of the length of the fluid collection assembly 100.

The opening 110 in the fluid impermeable barrier 102 may exhibit a width measured transverse to the longitudinal axis of the fluid collection assembly 100 that may be at least about 10% of the circumference of the fluid collection assembly 100, such as about 25% to about 50%, about 40% to about 60%, about 50% to about 75%, about 65% to about 85%, or about 75% to about 100% of the circumference of the fluid collection assembly 100. The opening 110 may exhibit a width that is greater than 50% of the circumference of the fluid collection assembly 100 since the vacuum (e.g., suction) through the conduit 134 pulls the fluid through the porous material 114 and into the conduit 134.

In some examples, the opening 110 may be vertically oriented (e.g., having a major axis parallel to the longitudinal axis of the fluid collection assembly 100). In some examples (not shown), the opening 110 may be horizontally oriented (e.g., having a major axis perpendicular to the longitudinal axis of the fluid collection assembly 100). In an example, the fluid impermeable barrier 102 may be configured to be attached to the patient, such as adhesively attached (e.g., with a hydrogel adhesive) to the patient. According to an example, a suitable adhesive is a hydrogel layer.

As previously discussed, the fluid impermeable barrier 102 may define fluid outlet 112 configured to remove bodily fluids from the chamber 108. The fluid outlet 112 is distinct from the opening 110 and the valve 122. In some examples, the fluid outlet 112 is sized to receive the conduit 134. The conduit 134 may be disposed in the chamber 108 via the fluid outlet 112. The fluid outlet 112 may be sized and shaped to form an at least substantially fluid tight seal against the conduit 134 or the at least one tube substantially preventing the bodily fluids from escaping the chamber 108.

The fluid impermeable barrier 102 may include markings thereon, such as one or more markings to aid a user in aligning the fluid collection assembly 100 on the patient. For example, a line on the fluid impermeable barrier 102 (e.g., opposite the opening 110) may allow a healthcare professional to align the opening 110 over the urethral opening of the patient. In examples, the markings may include one or more of alignment guide or an orientation indicator, such as a stripe or hashes. Such markings may be positioned to align the fluid collection assembly 100 to one or more anatomical features such as a pubic bone, etc.

As previously discussed, the fluid collection assembly 100 includes porous material 114 disposed in the chamber 108. The porous material 114 may cover at least a portion (e.g., all) of the opening 110. The porous material 114 is exposed to the environment outside of the chamber 108 through the opening 110. The permeable properties referred to herein may be wicking, capillary action, absorption, diffusion, or other similar properties or processes, and are referred to herein as “permeable” and/or “porous.” The porous material 114 may also wick the bodily fluids generally towards an interior of the chamber 108, as discussed in more detail below. The porous material 114 may include one or more of a fluid permeable membrane 136 or a fluid permeable support 138.

In an embodiment, at least a portion of the porous material 114 may be a wicking material configured to wick and/or enable the bodily fluids to move away from the opening 110, thereby preventing bodily fluids from escaping the chamber 108. The porous material may not include absorption of the bodily fluids into the porous material. Put another way, substantially no absorption of the bodily fluids into the porous material may take place after the wicking material is exposed to the bodily fluids. While no absorption is desired, the term “substantially no absorption” may allow for nominal amounts of absorption of the bodily fluids into the porous material (e.g., absorbency), such as about 30 wt % of the dry weight of the porous material, about 20 wt %, about 10 wt %, about 7 wt %, about 5 wt %, about 3 wt %, about 2 wt %, about 1 wt %, or about 0.5 wt % of the dry weight of the porous material. In an embodiment, the porous material 114 may be at least one of an absorbent material or adsorbent material instead of or in addition to being a wicking material.

The fluid collection assembly 100 may include the fluid permeable membrane 136 disposed in the chamber 108. The fluid permeable membrane 136 may cover at least a portion (e.g., all) of the opening 110. The fluid permeable membrane 136 may be composed to pull/push the bodily fluids away from the opening 110, thereby promoting fluid flow into the chamber 108, prevent fluid remaining on the vulva of the patient, and preventing the bodily fluids from escaping the chamber 108.

The fluid permeable membrane 136 may include any material that may be permeable to the bodily fluids. For example, the fluid permeable membrane 136 may include fabric, such as a gauze (e.g., a silk, linen, or cotton gauze), another soft fabric, or another smooth fabric. Forming the fluid permeable membrane 136 from gauze, soft fabric, and/or smooth fabric may reduce chaffing caused by the fluid collection assembly 100 and makes wearing the fluid collection assembly more comfortable. In an embodiment, the fluid permeable membrane 136 may define a plurality of perforations or may be continuous (e.g., does not define perforations). In an embodiment, the fluid permeable membrane 136 defines at least one hole that is configured to allow the valve 122 to extend through the fluid permeable membrane 136.

The fluid collection assembly 100 may include the fluid permeable support 138 disposed in the chamber 108. The fluid permeable support 138 is configured to support the fluid permeable membrane 136 and maintain the shape of the chamber 108 since the fluid impermeable barrier 102 and the fluid permeable membrane 136 may be formed from a relatively foldable, flimsy, or otherwise easily deformable material. For example, the fluid permeable support 138 may be positioned so the fluid permeable membrane 136 is disposed between the fluid permeable support 138 and the fluid impermeable barrier 102. The fluid permeable support 138 may support and maintain the position of the fluid permeable membrane 136 and the shape of the chamber 108. The fluid permeable support 138 may include any material that may be permeable to the bodily fluids, such as any of the fluid permeable membrane 136 materials disclosed above. For example, the fluid permeable membrane 136 material(s) may be utilized in a more dense or rigid form than in the fluid permeable membrane 136 when used as the fluid permeable support 138. The fluid permeable support 138 may be formed from any fluid porous material that is less deformable than the fluid permeable membrane 136. For example, the fluid permeable support 138 may include a porous polymer (e.g., nylon, polyester, polyurethane, polyethylene, polypropylene, etc.) structure (e.g., spun fibers such as spun nylon fibers) or a foam (e.g., an open cell foam). In some examples, the fluid permeable support 138 may be formed from a natural material, such as cotton, wool, silk, or combinations thereof. In such examples, the material may have a coating to prevent or limit absorption of the bodily fluids into the material, such as a water repellent coating. In some examples, the fluid permeable support 138 may be formed from fabric, felt, gauze, or combinations thereof.

In some examples, the fluid permeable membrane 136 may be omitted. For example, the porous material 114 may include only the fluid permeable support 138. In such examples, the bladder 118 may be positioned within the fluid permeable support 138 since, for instance, at least some materials of the support 138 disclosed herein are flexible enough to accommodate the shape and/or size changes discussed herein. In some examples, the fluid permeable support 138 may be optionally omitted from the fluid collection assembly 100 and the porous material 114 may only include the fluid permeable membrane 136. In such examples, the bladder 118 may be positioned within the fluid permeable membrane 136.

In an embodiment, the fluid permeable membrane 136 and/or the fluid permeable support 138 are wicking materials. In such an embodiment, the fluid permeable support 138 may have a greater ability to wick the bodily fluids than the fluid permeable membrane 136. In some examples, the wicking ability of the fluid permeable support 138 and the fluid permeable membrane 136 may be substantially the same. In an embodiment, the fluid permeable membrane 136 and/or the fluid permeable support 138 are non-wicking materials (e.g., absorbent and/or adsorbent materials).

In an embodiment, not shown, the fluid permeable membrane 136 and the fluid permeable support 138 may at least substantially completely fill the portions of the chamber 108 not occupied by the inflation device 116 and the conduit 134. In an embodiment, as shown in FIG. 1E, the fluid permeable membrane 136 and the fluid permeable support 138 may not substantially completely fill the portions of the chamber 108 not occupied by the inflation device 116 or the conduit 134. In such an embodiment, the fluid collection assembly 100 includes the fluid reservoir 140 disposed in the chamber 108.

The fluid reservoir 140 is a substantially unoccupied portion of the chamber 108. The fluid reservoir 140 may be defined between the fluid impermeable barrier 102 and at least one of the inflation device 116, the fluid permeable membrane 136, or the fluid permeable support 138. The bodily fluids in the chamber 108 may flow through the fluid permeable membrane 136 and/or fluid permeable support 138 to the fluid reservoir 140. The fluid reservoir 140 may retain of the bodily fluids. The bodily fluids in the chamber 108 may flow through the fluid permeable membrane 136 and/or fluid permeable support 138 and, optionally, to the fluid reservoir 140. The fluid impermeable barrier 102 may retain the bodily fluids in the fluid reservoir 140. The fluid reservoir 140 may be in a portion of the chamber 108 designed to be in a gravimetrically low point of the fluid collection assembly 100 when the fluid collection assembly 100 is worn.

FIGS. 2-4 are cross-sectional schematics and FIGS. 5 and 6 are side views of different fluid collection assemblies illustrating different techniques for controlling the expansion rate of the first and second regions of the bladders, according to different embodiments. Except as otherwise disclosed herein, the fluid collection assemblies illustrated in FIGS. 2-6 are the same or substantially similar to any of the fluid collection assemblies disclosed herein. For example, the fluid collection assemblies illustrated in FIGS. 2-6 may include a fluid impermeable barrier defining at least one opening, a chamber, and a fluid outlet. The fluid collection assemblies also include at least one porous material disposed in the chamber and at least one inflation device. The features of the fluid collection assemblies illustrated in FIGS. 2-6 may also be used in any fluid collection assemblies disclosed herein.

Referring to FIG. 2, the inflation device 216 of the fluid collection assembly 200 includes a bladder 218 and a valve 222. The bladder 218 includes one or more walls 217 defining an interior region 220. The bladder 218 includes a first region 224 and a second region 226. In the illustrated embodiment, the first and second regions 224, 226 are formed from the walls 217. The first region 224 and the second region 226 exhibit different thicknesses. The different thicknesses of the 224, 226 cause the first and second regions 224, 226 to exhibit different expansion rates when the bladder 218 switches from the first state to the second state.

The first region 224 exhibits a first thickness and an opposing portion of the second region 226 exhibits a second thickness that is different than the first thickness. For example, the first thickness may be greater than the second thickness, or vice versa, by at least about 1%, at least about 2%, at least about 3%, at least about 4%, at least about 5%, at least about 7.5%, at least about 10%, at least about 15%, at least about 20%, at least about 25%, at least about 30%, at least about 40%, at least about 50%, at least about 60%, at least about 70%, at least about 80%, at least about 90%, at least about 100%, at least about 125%, at least about 150%, at least about 200%, at least about 250%, at least about 300%, at least about 350%, at least about 400%, at least about 500%, or in ranges of about 1% to about 3%, about 2%, to about 4%, about 3% to about 5%, about 4% to about 7.5%, about 5% to about 10%, about 7.5% to about 15%, about 10% to about 20%, about 15% to about 25%, about 20% to about 40%, about 30% to about 50%, about 40% to about 60%, about 50% to about 70%, about 60% to about 80%, about 70% to about 90%, about 80% to about 100%, about 90% to about 125%, about 100% to about 150%, about 125% to about 200%, about 150% to about 250%, about 200% to about 300%, about 250% to about 350%, about 300% to about 400%, or about 350% to about 500%. As will be discussed in more detail below, the different between the first thickness and the second thickness may be selected based on the desired expansion rates of the opposing portions of the first and second regions 224, 226 which, in turn, affects the shape that the bladder 218 assumes when switching between the first and second states.

In an example, as illustrated, the first thickness is greater than the second thickness. The greater thickness of the first region 224 relative to the second region 226 causes the first region 224 to exhibit a smaller expansion rate than the second region 226. As such, the length of the first region 224 increases less than the length of the second region 226 when the bladder 218 switches from the first state to the second state which causes the bladder 218 to bend towards the first region 224. In other words, if the bladder 218 is initially straight, the increased thickness of the first region 224 causes the bladder 218 to form a curved shape with the first region 224 forming the concaved portion of the curved shape. In an example, the first thickness is less than the second thickness. The smaller thickness of the first region 224 relative to the second region 226 causes the first region 224 to exhibit an expansion rate that is greater than the second region 226. In other words, the length of the first region 224 increases more than the length of the second region 226 when the bladder 218 switches from the first state to the second state which causes the bladder 218 to bend towards the second region 226. In other words, if the bladder 218 is initially curved such that the first region 224 is concavely curved, the decreases thickness of the first region 224 causes the bladder 218 to form a more straight shape.

The shape that the bladder 218 forms when switching the bladder 218 between the first and second states may be controlled at least partially based on the thicknesses of the first and second regions 224, 226. In an embodiment, the bladder 218 may form a generally circular arc when each of the thickness of the first region 224 and the thickness of the second region 226 are substantially constant along the length of the bladder 218. In an embodiment, the thickness of at least one of the first region 224 or the second region 226 may be selectively varied which causes the expansion rate of different portions of the first region 224 and/or the second region 226 to vary. In an example, decreasing the thickness of a portion of the first region 224 relative to another portion of the first region 224 may cause the thinner portion of the first region 224 to exhibit a greater expansion rate than the thicker portion of the first region 224. In such an example, using the illustrated embodiment, the thinner portion of the first region 224 may bend more (e.g., exhibit a smaller average radius of curvature) than the thicker portion of the first region 224. In an example, decreasing the thickness of a portion of the second region 226 relative to another portion of the second region 226 may cause the thinner portion of the second region 226 to exhibit an expansion rate that is greater than the thicker portion of the second region 226.

Referring to FIG. 3, the inflation device 316 of the fluid collection assembly 300 includes a bladder 318 and a valve 322. The bladder 318 includes one or more walls 317 defining an interior region 320. The bladder 318 includes a first region 324 and a second region 326. In the illustrated embodiment, the first and second regions 324, 326 are formed from the walls 317. The inflation device 316 also includes at least one additional layer 342 attached to at least a portion of at least one of the first region 324 or the second region 326. The additional layer 342 is distinct from the walls 317.

The additional layer 342 effectively increases the thickness of the portion of the first region 324 and/or the second region 326 to which the additional layer 342 is adjacent. As such, similar to the different thicknesses discussed with regards to FIG. 2, the additional layer 342 may be used to control the shape that the bladder 318 assumes when switching the bladder 318 between the first and second states. In particular, the increased thickness caused by the additional layer 342 may decrease the expansion rate of the portion(s) of the bladder 318 to which the additional layer 342 is adjacent.

In an embodiment, the additional layer 342 may exhibit a Young's modulus (modulus of elasticity) that is greater than the walls 317 of the bladder 318. The increased Young's modulus of the additional layer 342 allows the additional layer 342 to exhibit the same effect as a thicker region of the walls 317 but at a smaller thickness. As such, the additional layer 342 may decrease the amount that the bladder 318 extends from the fluid impermeable barrier 302 than if bladder 318 included a region with increased thickness. The decreased profile of the bladder 318 may make the bladder 318 more comfortable to wear, especially with larger thighs since there is not significant space between the larger thighs in which to position the fluid collection assembly 300. In an example, the additional layer 342 includes a substantially inflexible fabric. In an example, the additional layer 342 includes a wire, a plate, a substantially inflexible polymer, a metal, or a composite.

The shape that the bladder 318 forms when switching the bladder 318 between the first and second states may be controlled based on which portions of the bladder 318 to which the additional layer 342 is adjacent and the Young's modulus of the additional layer 342. In an embodiment, the bladder 318 may form a generally circular arc when the additional layer 342 is adjacent to one or more of the first region 324 or the second region 326 along an entire length thereof. In an embodiment, the additional layer 342 is selectively positioned adjacent to only a portion of the length of one or more of the first region 324 or the second region 326. The portion(s) of the first region 324 and/or second region 326 to which the additional layer 342 is adjacent may exhibit an expansion rate that is less than portion(s) of the first regions 324 and/or second region 326 that are not spaced from the additional layer 342. In an embodiment, the additional layer 342 may be selectively formed form two or more portions, where at least two of the two or more portions exhibit different Young's moduli. Each of the two or more portions may extend along different lengths of the additional layer 342. For instance, the additional layer 342 may include a first portion exhibiting a first Young's modulus and a second portion exhibiting a second Young's modulus that is greater than the first Young's modulus. In such an instance, the portions of the bladder 318 adjacent to the first portion of the additional layer 342 may exhibit an expansion rate that is greater than the portions of the bladder 318 adjacent to the second portion of the additional layer 342.

Referring to FIG. 4, the inflation device 416 of the fluid collection assembly 400 includes a bladder 418 and a valve 422. The bladder 418 includes one or more walls 417. The walls 417 and a portion of the fluid impermeable barrier 402 defines an interior region 420 of the bladder 418. For example, the bladder 418 includes a first region 424 at least partially formed by the fluid impermeable barrier 402 and a second region 426 formed by the walls 417. The walls 417 may be attached to the fluid impermeable barrier 402 in a fluid tight manner such that inflation fluids present in the interior region 420 do not leak from the interior region 420. Inflating the bladder 418 will cause a tensile force to be applied to the portion of the fluid impermeable barrier 402 that defines the interior region 420, thereby causing the portion of the fluid impermeable barrier 402 that defines the interior region 420 to expand.

The fluid impermeable barrier 402 may exhibit an expansion rate that is different than the walls 417. In an example, the fluid impermeable barrier 402 may exhibit an expansion rate that is less than the walls 417. The fluid impermeable barrier 402 may exhibit the lower expansion rate because at least one of the fluid impermeable barrier 402 is thicker than the walls 417, the fluid impermeable barrier 402 exhibits a Young's modulus that is greater than the walls 417, or the portions of the fluid impermeable barrier 402 that do not define the interior region 420 apply a force to the first region 424 that limits expansion of the portions of the fluid impermeable barrier 402 that define the interior region 420. In an example, the fluid impermeable barrier 402 may exhibit an expansion rate that is greater than the walls 417. In such an example, the fluid impermeable barrier 402 may exhibit the larger expansion rate because at least one of the fluid impermeable barrier 402 is thinner than the walls 417 or the fluid impermeable barrier 402 exhibits a Young's modulus that is less than the walls 417.

Referring to FIG. 5, the fluid collection assembly 500 includes a fluid impermeable barrier 502 and at least one inflation device 516. The inflation device 516 includes a bladder 518 and at least one valve 522. The bladder 518 includes one or more walls 517 that completely define an interior region (not shown, obscured). The one or more walls 517 are attached to the fluid impermeable barrier 502 along substantially an entire length of the bladder 518 (attachment is illustrated schematically with a bold line). For example, as illustrated, the bladder 518 includes a first region 524 and a second region 526 and at least substantially all of the first region 524 is attached to the fluid impermeable barrier 502. Attaching substantially an entire length of the bladder 518 effectively increases the thickness of the portion of the bladder 518 that is attached to the fluid impermeable barrier 502. In other words, the fluid impermeable barrier 502 decreases the expansion rate of a surface of the bladder 518 that is attached to the fluid impermeable barrier 502 than if the portion of the bladder 518 was not attached to the fluid impermeable barrier 502.

The bladder 518 may be attached to the fluid impermeable barrier 502 using any suitable technique. For example, the bladder 518 may be attached to the fluid impermeable barrier 502 using an adhesive, radio frequency welding, ultrasonic welding, tape (e.g., double sided tape positioned between bladder 518 and the fluid impermeable barrier 502 or tape that wraps around at least a portion of the bladder 518 and is attached to the fluid impermeable barrier 502), or any other suitable attachment technique.

Referring to FIG. 6, the fluid collection assembly 600 includes a fluid impermeable barrier 602 and at least one inflation device 616. The inflation device 616 includes a bladder 618 and at least one valve 622. The bladder 618 includes one or more walls 617 that completely define an interior region (not shown, obscured). The one or more walls 617 are attached to the fluid impermeable barrier 602 along a portion of the length of the bladder 618 (attachment is illustrated schematically with a bold line). For example, the bladder 618 may include a first region 624 adjacent to the fluid impermeable barrier 602 and a second region 626 spaced from the first region 624. In such an example, the expansion rate of at least the first region 624 of the portion of the bladder 618 that is attached to the fluid impermeable barrier 602 is limited because the fluid impermeable barrier 602 effectively increases the thickness of such portions of the first region 624.

In an embodiment, two or more portions of the bladder 618 are attached to the fluid impermeable barrier 602. For example, as illustrated, two or more portions of the bladder 618 at or near the terminal ends of the bladder 618 are attached to the fluid impermeable barrier 602. The expansion rate of a surface of the bladder 618 that is adjacent to the fluid impermeable barrier 602 (i.e., the first portion 624) and is between two or more portions of the bladder 618 that are attached to the fluid impermeable barrier 602 is limited because the two or more portions that are attached to the bladder 618 exhibits a compressive force that limit the expansion rate. In other words, the surface of the bladder 618 that is adjacent to the fluid impermeable barrier 602 and is between two or more portions of the bladder 618 exhibits an expansion rate that is less than an opposing portion of the bladder 618.

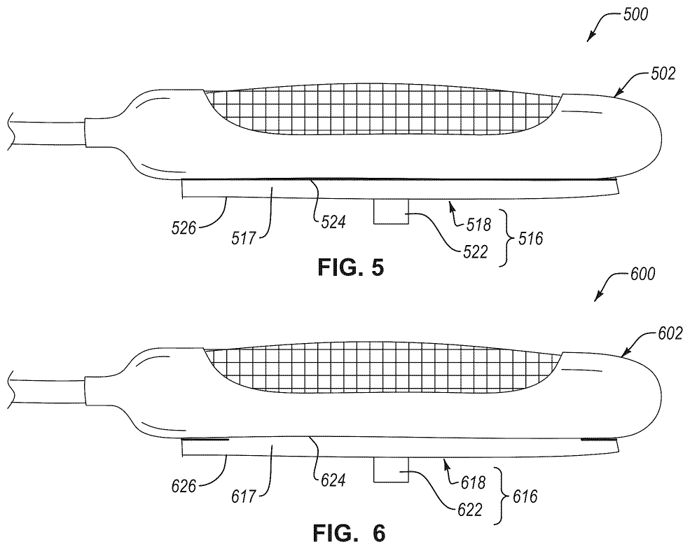

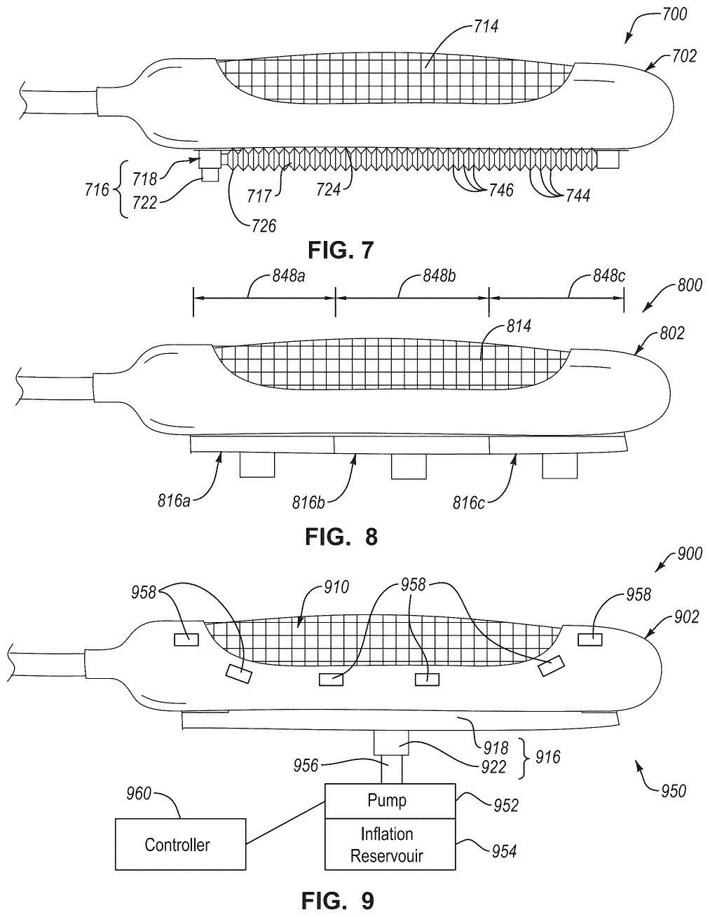

The embodiments illustrated and discussed in FIGS. 1A-6 include a bladder that exhibits a substantially uniform diameter. However, it is noted that any of the bladders disclosed herein may exhibit other shapes. For example, FIG. 7 is side view of a fluid collection assembly 700 that includes a “crinkle” or bellows shaped bladder 718, according to an embodiment. Except as otherwise disclosed herein, the fluid collection assembly 700 is the same or substantially similar to any of the fluid collection assemblies disclosed herein. For example, the fluid collection assembly 700 includes a fluid impermeable barrier 702, at least one porous material 714, and at least one inflation device 716.

The inflation device 716 includes a bladder 718 and at least one valve 722. The bladder 718 includes one or more walls 717 defining an interior region (not shown, obscured). The bladder 718 exhibits a “crinkle” structure (i.e., the bladder 718 is a crinkle shaped bladder). For example, the bladder 718 may include one or more peaks 744 (e.g., a plurality of circumferentially extending peaks 744 or a single helically extending peak 744). The bladder 718 also includes one or more valleys 746 (e.g., a plurality of circumferentially extending valleys 746 or a single helically extending valley 746) disposed between portions of the peaks 744 space apart along a longitudinal direction of the bladder 718. The peaks 744 and the valleys 746 allow the bladder 718 to bend when switching the bladder 718 from the first state to the second state. For example, adding inflation fluids to the bladder 718 may cause the distance between the peaks 744 on the second region 726 to increase while the distance between the peaks 744 on the first region 724 may decrease, remain constant, or increase at a rate that is less than the second region 726.

The inflation device 716 may be attached to the fluid impermeable barrier 702 using any suitable technique (e.g., adhesive, tape, ultrasonic welding, etc.) along at least a portion of a length thereof. In an example, only the terminal ends of the inflation device 716 (e.g., the terminal ends may not include the “crinkle structure”) may be attached to the fluid impermeable barrier 702. In an example, some or all of the peaks 744 may be attached to the fluid impermeable barrier 702. In an example, some or all of the valleys 746 may be attached to the fluid impermeable barrier 702. In an example, two or more of the terminal ends, at least some of the peaks 744, or at least some of the valleys 746 of the inflation device 716 are attached to the fluid impermeable barrier 702.

The embodiments illustrated in FIGS. 1A-7 illustrate that the fluid collection assemblies include a single inflation device. However, any of the fluid collection assemblies may include a plurality of inflation devices. FIG. 8 is a side view of a fluid collection assembly 800 that includes a plurality of inflation devices, according to an embodiment. Except as otherwise disclosed herein, the fluid collection assembly 800 may be the same or substantially similar to any of the fluid collection assemblies disclosed herein. For example, the fluid collection assembly 800 includes a fluid impermeable barrier 402 and at least one porous material 814.

As previously discussed, the fluid collection assembly 800 includes a plurality of inflation devices that may be independently inflated and/or deflated. For example, as illustrated, the fluid collection assembly includes a first inflation device 816a, a second inflation device 816b, and a third inflation device 816c. Each of the first inflation device 816a, the second inflation device 816b, and the third inflation device 816c may be the same or substantially similar to any of the inflation devices disclosed herein. For example, each of the first inflation device 816a, the second inflation device 816b, and the third inflation device 816c may a bladder having one or more walls defining and interior region and at least one valve. In an example, each of the first, second, and third inflation devices 816a, 816b, 816c are the same. In an example, at least two of the first, second, and third inflation devices 816a, 816b, 816c are different (e.g., exhibit different lengths, exhibit different expansion rates, limit the expansion rates thereof using different techniques, etc.).

Each of the first, second, and third inflation devices 816a, 816b, 816c are configured to control the shape of a corresponding portion of the fluid impermeable barrier 802. For example, the first inflation device 816a may be configured to control the shape of a first portion 848a of the fluid collection assembly 800, the second inflation device 816b may be configured to control the shape of a second portion 848b of the fluid collection assembly 800, and the third inflation device 816c may be configured to control the shape of a third portion 848c of the fluid collection assembly 800. As such, the first, second, and third inflation devices 816a, 816b, 816c allow for more control of the shape of the fluid collection assembly 800 that if the fluid collection assembly 800 included a single inflation device. In an example, one or two of the first, second, or third inflation devices 816a, 816b, 816c may be switched from the first state to the second state while the remainder of the first, second, or third inflation devices 816a, 816b, 816c remain in the first state. The inflation device(s) that are switched to the second state cause the corresponding portions of the fluid collection assembly to change shape while the inflation device(s) that remain in the first state cause the corresponding portions of the fluid collection assembly to remain in the original shape thereof. In an example, each of the first, second, or third inflation devices 816a, 816b, 816c are switched from the first state to the second state. In such an example, one or two of the first, second, or third inflation devices 816a, 816b, 816c may include changing (e.g., reducing) an average radius of curvature of a corresponding portion of the fluid collection assembly 800 more than the remaining one or two of the first, second, or third inflation devices 816a, 816b, 816c.

In an embodiment, the fluid collection assembly 800 may include two inflation devices or four or more inflation devices. In other words, the third inflation device 816c may be omitted from the fluid collection assembly 800 or the fluid collection assembly 800 may include one or more additional inflation devices in addition to the first, second, and third inflation devices 816a, 816b, 816c. In an embodiment, as shown, the first, second, and third portions 848a, 848b, 848c of the fluid impermeable barrier 802 may not overlap. However, it is noted that two or more of the first, second, or third portions 848a, 848b, 848c may overlap such that a shape of such overlapped portions may be controlled by two different inflation devices.

FIG. 9 is a schematic side view of a fluid collection system 950 that is configured to control the shape of a fluid collection assembly 900, according to an embodiment. The system 950 includes a fluid collection assembly 900. Except as otherwise disclosed herein, the fluid collection assembly 900 is the same or substantially similar to any of the fluid collection assemblies disclosed herein. For example, the fluid collection assembly 900 may include a fluid impermeable barrier 902 and at least one inflation device 916. The inflation device 916 includes a bladder 918 defining an interior region (not shown, obscured) and a valve 922.

The system 950 also includes at least one pump 952 and at least one inflation fluid reservoir 954 configured to store one or more inflation fluids therein. The inflation fluid reservoir 954 may include a tank that stores the inflation fluids therein or may be the atmosphere (e.g., the inflation fluids are atmospheric air). The pump 952 and the inflation fluid reservoir 954 are in fluid communication to the inflation device 916 via at least one conduit 956. For example, the conduit 956 may extend from the pump 952 and/or the inflation fluid reservoir 954 to a valve 922 of the inflation device 916. In an embodiment, the pump 952 may be configured to provide the inflation fluids to the inflation device 916 from the inflation fluid reservoir 954. The inflation fluids provided to the inflation device 916 may flow through the valve 922 into the interior region defined by the bladder 918 to switch the bladder 918 from the first state to the second state. As such, the pump 952 and the inflation fluid reservoir 954 may change the shape of the fluid collection assembly 900 from a first (e.g., initial) shape to a second shape. In an embodiment, the pump 952 and the inflation fluid reservoir 954 may be configured to remove the inflation fluids from the inflation device 916 instead of or in addition to providing the inflation fluids to the inflation device 916. In such an embodiment, the pump 952 may provide a suction force that removes at least some of the inflation fluids from the interior region of the bladder 918 which switches the bladder 918 from the second state to the first state. Switching the bladder 918 from the second state to the first state may change the fluid collection assembly 900 from the second shape to the first state. The inflation fluids removed from the interior region of the bladder may be deposited in the inflation fluid reservoir 954.

In an embodiment, the system 950 may be controlled responsive to receiving input from a user of the system 950 (e.g., a medical practitioner or a patient). The input may include verbal commands, manipulating one or more actuators (e.g., buttons, levers, etc.), or one or more electronic instructions sent from a computer. The pump 952 and the inflation fluid reservoir 954 may provide and/or remove inflation fluids from the inflation device 916 responsive to receiving the one or more inputs. In an embodiment, the system 950 may be at least partially controlled without receiving one or more inputs from the user of the system 950. In such an embodiment, the system 950 may include one or more sensor 958 that are configured to detect contact between a portion of the fluid collection assembly 900 and the patient. For example, the sensors 958 may include an array of contact sensors arranged around the opening 910 which allows the sensors 958 to detect whether one or more gaps are formed between the portions of the fluid impermeable barrier 902 that define the opening 910. The sensors 958 may be communicably coupled to a controller 960 (e.g., computer) and configured to transmit one or more signals to the control electric circuitry 960. The signals transmitted from the sensors 958 to the control electric circuitry 960 may include the characteristics detected by the sensors 958. The control electric circuitry 960 may control the pump 952 responsive to receiving the one or more signals from the sensors 958. For example, the control electric circuitry 960 may cause the pump 952 to add or remove inflation fluids from the inflation device 916 when the sensor 958 indicate that there are gaps between the fluid impermeable barrier 902 and the patient.

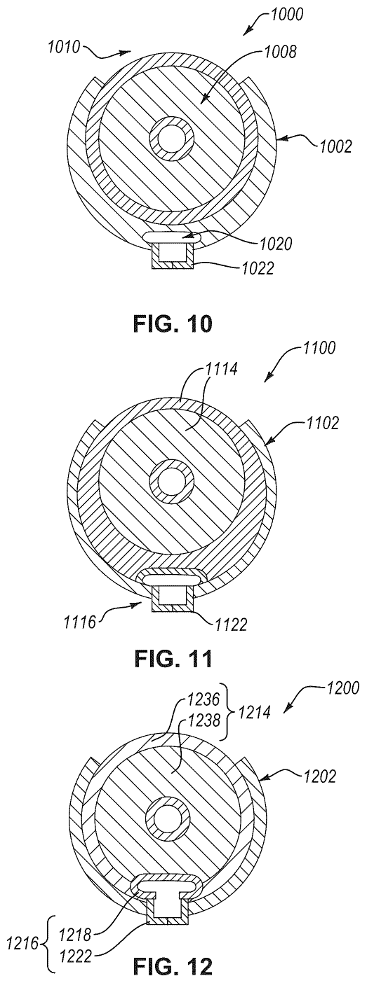

The embodiments illustrated in FIGS. 1A-9 illustrate the inflation device being disposed on an outer surface of the fluid impermeable barrier. However, it is noted that the inflation devices of any of the fluid collection assemblies disclosed herein may be positioned at locations other than the outer surface of the fluid impermeable barrier. FIGS. 10-12 are cross-sectional schematics of fluid collection assemblies having at least one inflation device positioned at locations other than an outer surface of the fluid impermeable barrier, according to different embodiments. Except as otherwise disclosed herein, the fluid collection assemblies illustrated in FIGS. 10-12 are the same or substantially similar to any of the fluid collection assemblies disclosed herein. For example, the fluid collection assemblies may each include a fluid impermeable barrier, at least one porous material, at least one inflation device, and a conduit. The inflation device may include a bladder and at least one valve. The bladder may include one or more walls at least partially defining an interior region. It is noted that the bladders are generally illustrated as being generally positioned between the conduit and a distal surface of the fluid impermeable barrier thereby minimizing the likelihood that switching the bladder from the first state to the second state significantly increases a maximum width of the fluid collection assembly for reasons previously discussed. However, it is noted that the bladders may be positioned between the conduit and a lateral surface of the fluid impermeable barrier.

Referring to FIG. 10, the fluid collection assembly 1000 includes a fluid impermeable barrier 1002. The fluid impermeable barrier 1002 forms the at least a portion of the inflation device (e.g., the fluid impermeable barrier 1002 and at least a portion of an inflation device are integrally formed together). The impermeable barrier 1002 may exhibit any of the properties and functions as any of the fluid impermeable barriers and inflation devices disclosed herein. For example, the impermeable barrier 1002 may define a chamber 1008, at least one opening 1010, and a fluid outlet and may be configured to prevent bodily fluids from leaking from the chamber 1008. The impermeable barrier 1002 may also include a bladder defining an interior region 1020. The impermeable barrier 1002 is configured to switch between one or more states (e.g., a first state and a second state) by adding or removing inflation fluids to the interior region 220 using a valve 1022.

Referring to FIG. 11, the fluid collection assembly 1100 includes a fluid impermeable barrier 1102 and at least one porous material 1114. The fluid collection assembly 1100 also includes at least one inflation device 1116 that is distinct from the fluid impermeable barrier 1102. At least a portion of the inflation device 1116 (e.g., at least the bladder 1118) is positioned between the fluid impermeable barrier 1102 and the porous material 1114. The fluid impermeable barrier 1102 may protect the inflation device 1116 from objects that may puncture the inflation device 1116. The fluid impermeable barrier 1102 may define an aperture through which the valve 1122 may extend. The aperture may be configured to form a fluid tight seal against the valve 1122 to prevent leaks between the aperture and the valve 1122.

Referring to FIG. 12, the fluid collection assembly 1200 includes a fluid impermeable barrier 1202 and at least one porous material 1214. The fluid collection assembly 1200 also include at least one inflation device 1216. At least a portion of the inflation device 1216 (e.g., at least the bladder 1218) is positioned within the porous material 1214. For example, when the porous material 1214 includes a fluid permeable membrane 1236 and a fluid permeable support 1238, at least a portion of the inflation device 1216 may be disposed between the fluid permeable membrane 1236 and the fluid permeable support 1238. As such, the fluid impermeable barrier 1202 and at least a portion of the porous material 1214 may protect the inflation device 1216 from objects that may puncture the inflation device 1216. The fluid impermeable barrier 1202 and at least a portion of the porous material 1214 may define an aperture through which the valve 1222 may extend. The aperture may be configured to form a fluid tight seal against the valve 1222 to prevent leaks between the aperture and the valve 1222.

It is noted that the inflation devices disclosed herein may have positions other that the positions illustrated in FIGS. 1A-12. For example, the inflation devices may be positioned within the fluid permeable membrane, within the fluid permeable support, between the fluid permeable support and the conduit, within the conduit, or integrally formed with the conduit.

In an embodiment, the conduit of any of the fluid collection assemblies disclosed herein (e.g., conduit 134 of FIGS. 1A-1E) may exhibit an initial shape when the conduit is in its relaxed state. The conduit is in its relaxed state when no external forces, such as forces caused by an inflation device (e.g., the inflation device is in a deflated state), are applied to the conduit. The initial shape of the conduit may be a generally cylindrical shape (e.g., the conduit is straight) or a slightly curved generally cylindrical shape. Changing the shape of the inflation device, such as switching the bladder of the inflation device from the first state to the second state, may apply an external force to the conduit that causes the conduit to change the shape thereof. However, the conduit may resist changing the shape thereof when the external force are applied to the conduit which causes the conduit to apply a normal force that is opposite the external force. The normal force from the conduit may cause the conduit to compress a portion of the porous material and/or may cause the formation of detrimental voids in the chamber.

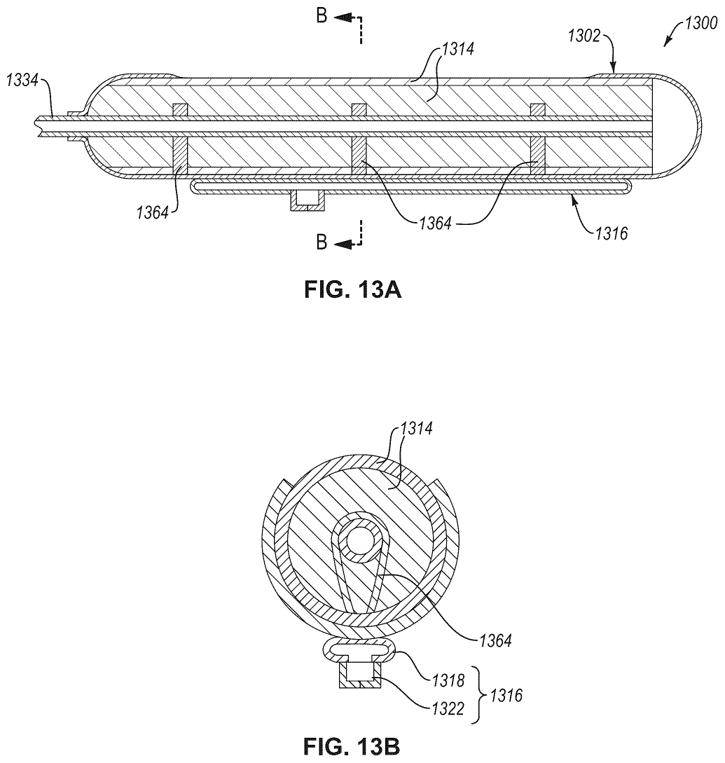

The fluid collection assemblies disclosed herein may include one or more structures that are configured to force the conduit to exhibit the desired shape change and minimize the normal force that is applied to the porous material. FIG. 13A is a cross-sectional view of a fluid collection assembly 1300, according to an embodiment. FIG. 13B is a cross-sectional view of the fluid collection assembly 1300 taken along plane B-B of FIG. 13A, according to an embodiment. Except as otherwise disclosed herein, the fluid collection assembly 1300 is the same or substantially similar at any of the fluid collection assemblies disclosed herein. For example, the fluid collection assembly 1300 includes a fluid impermeable barrier 1302, a porous material 1314, a conduit 1334, and at least one inflation device 1316. In the illustrated embodiment, the fluid collection assembly 1300 is illustrated as being substantially similar to the fluid collection assembly 100 shown in FIGS. 1A-1E. However, as previously discussed, the fluid collection assembly 1300 may be substantially similar to any of the fluid collection assemblies disclosed herein.

The fluid collection assembly 1300 includes at least one brace 1364 that is configured to force the conduit 1334 to exhibit a shape change that corresponds more closely to the shape change of the inflation device 1316 than if the fluid collection assembly 1300 did not include the brace 1364. The brace 1364 also minimizes the normal force applied from the conduit 1334 to the porous material 1314. The brace 1364 is connected to the inflation device 1316 or a component of the fluid collection assembly 1300 that is connected to the inflation device 1316 (e.g., the fluid impermeable barrier 1302 when the inflation device 1316 is attached to an exterior surface of or embedded within the fluid impermeable barrier 1302). The brace 1364 extends from the inflation device 1316 or the component of the fluid collection assembly 1300 that is connected to the inflation device 1316 to the conduit 1334. For example, the brace 1364 may extend around the conduit 1334, as shown in FIG. 13B. It is noted that the brace 1364 may be attached to the conduit 1334 using other suitable techniques, such as with an adhesive. To allow the brace 1364 to extend to the conduit 1334, one or more components of the fluid collection assembly 1300 (e.g., the porous material 1314) may include one or more slits formed therein through which the braces 1364 extend. The brace 1364 may be configured to transfer a shape change in the inflation device 1316 to the conduit 1334 such that the conduit 1334 exhibits a shape change that substantially corresponds to the shape change of the inflation device 1316.

The fluid collection assembly 1300 may include any suitable number of braces 1364, such as 1, 2, 3, 4, 5, 6, 7, 8, 9, 10, or greater than 10 braces 1364. Generally increasing the number of braces 1364 causes the conduit 1334 to more accurately correspond to the shape of the inflation device 1316 and decreases the normal force that is applied to the porous material 1314.

It is noted that the braces 1364 may not always cause the conduit 1334 to exhibit the same shape change as the inflation device 1316. For example, when the braces 1364 are looped around and are not attached to the conduit 1334 (as shown), the braces 1364 may only cause the conduit 1334 to exhibit the shape change of the inflation device 1316 when the braces 1364 pull (e.g., the braces 1364 are in tension) the conduit 1334 towards the inflation device 1316 but not when the braces 1364 push (e.g., the braces 1364 are in compression) the conduit 1334 away from the inflation device 1316.

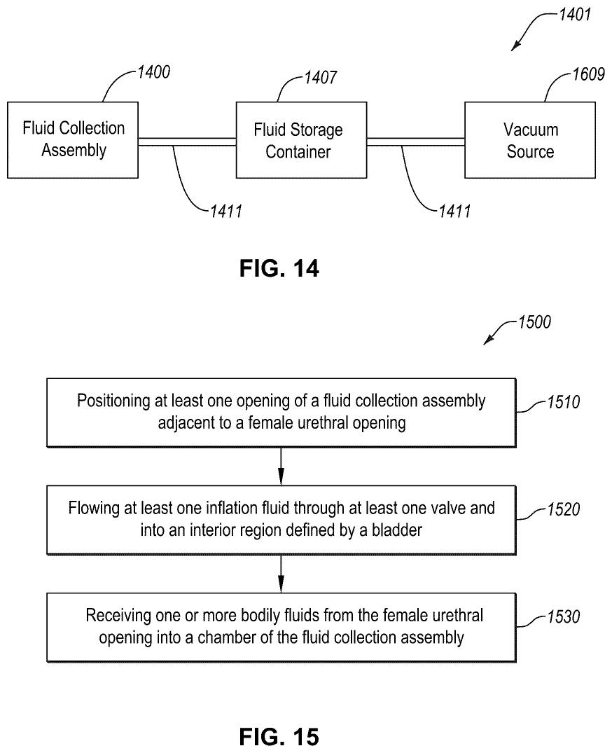

FIG. 14 is a block diagram of a system 1401 for fluid collection, according to an embodiment. The system 1401 includes a fluid collection assembly 1400, a fluid storage container 1407, and a vacuum source 1409. The fluid collection assembly 1400, the fluid storage container 1407, and the vacuum source 1409 may be fluidly coupled to each other via one or more conduits 1411. For example, fluid collection assembly 1400 may be operably coupled to one or more of the fluid storage container 1407 or the vacuum source 1409 via the conduit 1411. Fluid (e.g., urine or other bodily fluids) collected in the fluid collection assembly 1400 may be removed from the fluid collection assembly 1400 via the conduit 1411 which protrudes into the fluid collection assembly 1400. For example, an inlet of the conduit 1411 may extend into the fluid collection assembly 1400, such as to a fluid reservoir therein. The outlet of the conduit 1411 may extend into the fluid collection assembly 1400 or the vacuum source 1409. Suction force may be introduced into the chamber of the fluid collection assembly 1400 via the inlet of the conduit 1411 responsive to suction (e.g., vacuum) force applied at the outlet of the conduit 1411.

The suction force may be applied to the outlet of the conduit 1411 by the vacuum source 1409 either directly or indirectly. The suction force may be applied indirectly via the fluid storage container 1407. For example, the outlet of the conduit 1411 may be disposed within the fluid storage container 1407 and an additional conduit 1411 may extend from the fluid storage container 1407 to the vacuum source 1409. Accordingly, the vacuum source 1409 may apply suction to the fluid collection assembly 1400 via the fluid storage container 1407. The suction force may be applied directly via the vacuum source 1409. For example, the outlet of the conduit 1411 may be disposed within the vacuum source 1409. An additional conduit 1411 may extend from the vacuum source 1409 to a point outside of the fluid collection assembly 1400, such as to the fluid storage container 1407. In such examples, the vacuum source 1409 may be disposed between the fluid collection assembly 1400 and the fluid storage container 1407.

The fluid collection assembly 1400 may be similar or identical to any of the fluid collection assemblies disclosed herein in one or more aspects. The fluid collection assembly 1400 may be shaped and sized to be positioned adjacent to a female urethral opening. For example, the fluid collection assembly 1400 may include a fluid impermeable barrier at least partially defining a chamber (e.g., interior region) of the fluid collection assembly 1400. The fluid impermeable barrier also defines at least one opening extending therethrough from the external environment. The opening may be positioned adjacent to the female urethral opening. The fluid collection assembly 1400 may include porous material disposed in the chamber, such as one or more of a fluid permeable support and a fluid permeable membrane. The fluid collection assembly 1400 includes at least one inflation device on or incorporated in one or more components thereof. The inflation device include a bladder that is configured to change a shape thereof when the bladder is switched between the first and second states. The conduit 1411 may extend into the fluid collection assembly 1400 at a first end (e.g., proximal) region, through one or more of the fluid impermeable barrier or the porous material to a second end region of the fluid collection assembly 1400. The conduit 1411 includes an inlet and an outlet, the outlet being fluidly coupled to the fluid storage container and the inlet being positioned in a portion of the chamber selected to be at a gravimetrically low point of the fluid collection assembly when worn.

The fluid storage container 1407 is sized and shaped to retain a fluid therein. The fluid storage container 1407 may include a bag (e.g., drainage bag), a bottle or cup (e.g., collection jar), or any other enclosed container for storing bodily fluid(s) such as urine. In some examples, the conduit 1411 may extend from the fluid collection assembly 1400 and attach to the fluid storage container 1407 at a first point therein. An additional conduit 1411 may attach to the fluid storage container 1407 at a second point thereon and may extend and attach to the vacuum source 1409. Accordingly, a vacuum (e.g., suction) may be drawn through fluid collection assembly 1400 via the fluid storage container 1407. Fluid, such as urine, may be drained from the fluid collection assembly 1400 using the vacuum source 1409.

The vacuum source 1409 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 1409 may provide a vacuum or suction to remove fluid from the fluid collection assembly 1400. In some examples, the vacuum source 1409 may be powered by one or more of a power cord (e.g., connected to a power socket), one or more batteries, or even manual power (e.g., a hand operated vacuum pump). In some examples, the vacuum source 1409 may be sized and shaped to fit outside of, on, or within the fluid collection assembly 1400. For example, the vacuum source 1409 may include one or more miniaturized pumps or one or more micro pumps. The vacuum sources 1409 disclosed herein may include one or more of a switch, a button, a plug, a remote, or any other device suitable to activate the vacuum source 1409.

FIG. 15 is a flow diagram of a method 1500 to collect fluid, according to an embodiment. The method 1500 of collecting fluid may utilize use any of the fluid collection assemblies and/or fluid collection systems disclosed herein. The method 1500 may include act 1510, which recites “positioning at least one opening of a fluid collection assembly adjacent to a female urethral opening.” Act 1510 may be followed by act 1520, which recites “flowing at least one inflation fluid through at least one valve and into an interior region defined by a bladder.” Act 1520 may be followed by act 1530, which recites “receiving one or more bodily fluids from the female urethral opening into a chamber of the fluid collection assembly.”

Acts 1510, 1520, 1530 of the method 1500 are for illustrative purposes. For example, the act 1510, 1520, 1530 of the method 1500 may be performed in different orders, split into multiple acts, modified, supplemented, or combined. In an example, one or more of the acts 1510, 1520, 1530 of the method 1500 may be omitted from the method 1500. Any of the acts 1510, 1520, or 1530 may include using any of the fluid collection assemblies or systems disclosed herein.

Act 1510 recites “positioning at least one opening of a fluid collection assembly adjacent to a female urethral opening.” The act 1510 of positioning the opening of a fluid collection assembly adjacent to a female urethral opening may include utilizing any of the fluid collection assemblies or systems disclosed herein. In some examples, act 1510 may include positioning the opening of a fluid collection assembly such that the fluid permeable membrane of the female fluid collection assembly abuts or is positioned proximate to the female urethral opening. In some examples, positioning an opening of a fluid collection assembly adjacent to a female urethral opening may include positioning the opening over the female urethral opening, such as positioning a longitudinally extending opening of the fluid collection assembly over the female urethral opening.

Act 1520 recites “flowing at least one inflation fluid through at least one valve and into an interior region defined by a bladder.” Flowing the inflation fluid into the interior region may switch the bladder between a first state and at least a second state which, in turn, shapes the bladder and at least a portion of the rest of the fluid collection assembly between a first (e.g., initial) shape and a second shape. For example, switching the bladder from the first state to the second state may include shaping a female fluid collection assembly to contour to the anatomy around the urethral opening. In some embodiments, switching the bladder from the first state to the second state may include forming the (e.g., a longitudinal shape of the) fluid collection assembly into an arcuate shape conforming to the perineal region of the patient. For example, switching the bladder from the first state to the second state may include forming the fluid collection assembly into an arcuate shape conforming to the vaginal and perineal region of a patient.

Act 1530 recites, “receiving one or more bodily fluids from the female urethral opening into a chamber of the fluid collection assembly.” In some examples, receiving bodily fluids from the female urethral opening into a chamber of the fluid collection assembly includes receiving the bodily fluids through the opening of the fluid collection assembly. Receiving bodily fluids from the female urethral opening into a chamber of the fluid collection assembly may include wicking the bodily fluids away from the opening using porous material, such as via a fluid permeable membrane and a fluid permeable support. Receiving bodily fluids from the female urethral opening into a chamber of the fluid collection assembly may include flowing the bodily fluids towards a portion of the chamber that is fluidly coupled to an inlet of a conduit in fluid communication a vacuum source. For instance, receiving bodily fluids from the female urethral opening into a chamber of the fluid collection assembly may include flowing the bodily fluids to a substantially unoccupied portion of the chamber (e.g., a fluid reservoir), to a gravimetrically low point of the chamber, etc., such as via gravity, wicking, or suction force. In some examples, wicking the bodily fluids into the chamber via the fluid permeable membrane and fluid permeable support may include wicking urine into a fluid reservoir in the fluid collection assembly.

The method 1500 may include applying suction with a vacuum source effective to suction the bodily fluids from the chamber via a conduit disposed therein and fluidly coupled to the vacuum source may include using any of the vacuum sources disclosed herein. Applying suction with a vacuum source may include activating the vacuum source (e.g., suction device) in fluid communication with the inlet of the conduit in the fluid collection assembly. In some examples, activating the vacuum source in fluid communication with the inlet of the conduit in the fluid collection assembly may include supplying power to the vacuum source by one or more of flipping an on/off switch, pressing a button, plugging the vacuum source into a power outlet, putting batteries into the vacuum source, etc. In some examples, the vacuum source may include a hand operated vacuum pump and applying suction with a vacuum source may include manually operating the hand operated vacuum pump effective to suction the bodily fluids from the chamber via the conduit disposed therein that is fluidly coupled to the vacuum source.

In some examples, applying suction with a vacuum source effective to suction the bodily fluids from the chamber via a conduit disposed therein and fluidly coupled to the vacuum source may be effective to remove at least some bodily fluids (e.g., urine) from the chamber (e.g., interior region) of the fluid collection assembly. In some examples, applying suction with a vacuum source effective to suction the bodily fluids from the chamber via a conduit disposed therein and fluidly coupled to the vacuum source may be effective to transfer at least some of the bodily fluids from the chamber to a fluid storage container (e.g., a bottle or bag), such as from one or more of a reservoir, fluid permeable support, or fluid permeable membrane.

In some examples, the vacuum source (e.g., suction device) may be disposed on or within the fluid collection assembly and applying suction with the vacuum source may include activating the vacuum source. In some examples, the vacuum source may be spaced from the fluid collection assembly and applying suction with the vacuum source may include activating the vacuum source.

In some examples, applying suction with a vacuum source effective to suction the bodily fluids from the chamber via a conduit disposed therein and fluidly coupled to the vacuum source may include detecting moisture in the chamber (e.g., via one or more moisture sensors) and responsive thereto, activating the vacuum source to provide suction in the chamber. The control of the vacuum source responsive to the signals indicating that moisture or a level thereof is present in the chamber may be automatic, such as via a controller (e.g., computer programmed to perform the operation), or may merely provide an indication that a level of moisture is present that may necessitate removal of fluid from the chamber of the fluid collection assembly. In the latter case, a wearer may receive the indication (e.g., from the controller) and activate the vacuum pump manually.

In an example, the method 1500 may include collecting the bodily fluids that are removed from the fluid collection assembly, such as into a fluid storage container that is spaced from the fluid collection assembly and fluidly coupled to the conduit. The fluid storage container may include any of the fluid storage containers disclosed herein.

The fluid collection assemblies disclosed herein are configured to collect one or more bodily fluids from a female urethral opening. However, it is noted that any of the concepts disclosed herein, such as inflation devices, may be configured to collect one or more bodily fluids from a male urethral opening (e.g., penis). Examples of fluid collection assemblies that are configured to collected bodily fluids from a male urethral opening and methods of using such fluid collection assemblies are disclosed in International Application No. PCT/US20/42262 filed on Jul. 14, 2020, U.S. patent application Ser. No. 14/433,773 filed on Apr. 3, 2020, and U.S. Provisional Patent Application No. 63/047,374 filed on Jul. 2, 2020, the disclosure of each of which is incorporated herein, in its entirety, by this reference.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. (canceled)

2. A fluid collection assembly, comprising:
a fluid impermeable barrier defining a chamber, at least one opening, and at least one fluid outlet, the fluid impermeable barrier including at least one proximal surface defining the at least one opening and at least one distal surface opposite the proximal surface;
at least one porous material disposed in the chamber; and
at least one inflation device including:
at least one extender extending from the fluid impermeable barrier; and
at least one valve configured to selectively permit at least one inflation fluid to flow into and out of the at least one extender to switch the at least one extender between the first state and at least the second state, the at least one valve including a two-way valve.

3. The fluid collection assembly of claim 2, wherein the fluid impermeable barrier exhibits a generally cylindrical shape.

4. The fluid collection assembly of claim 2, wherein the at least one porous material includes a wicking material that exhibits substantially no absorption of bodily fluids.

5. The fluid collection assembly of claim 2, wherein the at least one extender is positioned external to the chamber and coupled to the at least one distal surface of the fluid impermeable barrier.

6. The fluid collection assembly of claim 5, wherein the at least one valve is extends from a portion of the at least one extender opposite the at least one distal surface of the fluid impermeable barrier.

7. The fluid collection assembly of claim 2, wherein the at least one extender includes one or more walls defining at least one interior region, the at least one extender configured to switch between a first state and at least a second state, wherein a volume of the at least one interior region is greater when the bladder is in the second state than when the bladder is in the first state.

8. The fluid collection assembly of claim 7, wherein the inflation device includes a single extender and the at least one extender defines a single interior region.

9. The fluid collection assembly of claim 2, wherein switching the bladder between the first state and the second state changes a curvature of at least a portion of the fluid impermeable barrier.

10. The fluid collection assembly of claim 2, wherein the at least one inflation device includes a first region adjacent to the at least one distal surface of the fluid impermeable barrier and a second region opposite the first region, and wherein an expansion rate of the first region is less than an expansion rate of the second region.

11. The fluid collection assembly of claim 10, wherein the at least one inflation device includes at least one additional layer attached to at least a portion of the first region.

12. The fluid collection assembly of claim 10, wherein at least a portion of the first region exhibits a thickness that is greater than an opposing portion of the second region.

13. The fluid collection assembly of claim 10, wherein at least a portion of the first region is directly attached to the fluid impermeable barrier.

14. The fluid collection assembly of claim 2, wherein the fluid impermeable barrier includes at least one lateral surface extending between the at least one proximal surface and the at least one distal surface, and wherein the at least one inflation device is not adjacent to the at least one lateral surface or the at least one proximal surface.

15. The fluid collection assembly of claim 2, wherein the fluid impermeable barrier includes at least one lateral surface extending between the at least one proximal surface and the at least one distal surface, and wherein the at least one inflation device is adjacent to the at least one lateral surface.

16. The fluid collection assembly of claim 2, wherein the at least one inflation device includes a plurality of inflation devices adjacent to the at least one distal surface.

17. The fluid collection assembly of claim 2, wherein at least a portion of the at least one extender extends into the chamber.

18. The fluid collection assembly of claim 2, wherein a maximum width of the fluid collection assembly is unchanged when switching the at least one extender between the first state and the second state.

19. A system, comprising:
a fluid collection assembly including:
a fluid impermeable barrier defining a chamber, at least one opening, and at least one fluid outlet, the fluid impermeable barrier including at least one proximal surface defining the at least one opening and at least one distal surface opposite the proximal surface;
at least one porous material disposed in the chamber; and
at least one inflation device including:
at least one extender extending from the fluid impermeable barrier; and
at least one valve configured to selectively permit at least one inflation fluid to flow into and out of the extender to switch the extender between the first state and at least the second state, the at least one valve including a two-way valve;
a fluid storage container; and
a vacuum source;
wherein the chamber of the fluid collection assembly, the fluid storage container, and the vacuum source are in fluid communication with each other via one or more conduits.

20. The system of claim 19, further comprising at least one pump in fluid communication with the at least one valve, the at least one pump configured to provide at least one inflation fluid to switch the bladder from the first state to the second state.

21. The system of claim 20, further comprising one or more sensors configured to detect contact between the fluid collection assembly and a patient, the one or more sensors communicably coupled to a control electric circuitry, the control electric circuitry configured to control the pump responsive to receiving one or more signal from the one or more sensors.
